(19) United States
(12) Patent Application Publication
(10) Pub. No.: US 2006/0069149 A1
(43) Pub. Date: Mar. 30, 2006

(54) Thiadibenzoazulene derivatives for the treatment of inflammatory diseases

(75) Inventors: Mladen Mercep, Zagreb (HR); Milan Mesic, Zagreb (HR); Dijana Pesic, Sibenik (HR); Iva Benko, Velika Gorica (HR)

Correspondence Address: DARBY & DARBY P.C., P. O. BOX 5257, NEW YORK, NY, 10150-5257, US

(73) Assignee: Pliva-Istrazivacki Institut d.o.o., Zagreb (HR)

(21) Appl. No.: 11/221,414

(22) Filed: Sep. 6, 2005

Related U.S. Application Data
(63) Continuation of application No. PCT/HR04/00005, filed on Mar. 5, 2004.

(30) Foreign Application Priority Data
Mar. 6, 2003 (HR) P20030160A

(51) Int. Cl. A61K 31/381; C07D 495/02

(52) U.S. Cl. 514/443; 549/12; 549/42

(57) ABSTRACT

The present invention relates to (a) novel 1-thiadibenzoazulene derivatives of the formula (I), (b) to their pharmacologically acceptable esters, salts and solvates, (c) to processes and intermediates for the preparation thereof, (d) to a process for preparing pharmaceutical formulations for the treatment of inflammatory diseases and conditions and (e) to the use thereof in the treatment of inflammatory diseases and conditions in humans and animals. These compounds inhibit tumour necrosis factor-α (TNF-α) production and interleukin-1 (IL-1) production and exhibit an analgetic action.

### TECHNICAL FIELD

The present invention relates to (a) novel 1-thiadibenzoazulene derivatives of the formula (I), (b) to their pharmacologically acceptable esters, salts and solvates, (c) to processes and intermediates for the preparation thereof, (d) to a process for preparing pharmaceutical formulations for the treatment of inflammatory diseases and conditions and (e) to the use thereof in the treatment of inflammatory diseases and conditions in humans and animals. These compounds inhibit the tumour necrosis factor-α (TNF-α) production and interleukin-1 (IL-1) production and exhibit an analgetic action.

### PRIOR ART

Some 1,3-diaza-dibenzoazulene derivatives and salts thereof are well known as a novel class of compounds having an antiinflammatory action (U.S. Pat. No. 3,711,489, U.S. Pat. No. 4,198,421, CA 967,573 and HR Patent Application No. 20020453A). In the literature, from the class of 1-thia-dibenzoazulenes there are disclosed derivatives substituted in 2-position with methyl, methyl-ketone, nitro group or with carboxylic group derivatives (Cagniant P G, C. R. Hebd. Sceances Acad. Sci., 1976, 283:683-686), or with alkyloxy groups (WO 01/878990), which also possess a strong antiinflammatory action. However, according to our knowledge and to available literature data, 1-thia-dibenzoazulenes of the present invention having aminoalkyloxy substituents on benzene rings are not known. It is not known either that such compounds could possess an antiinflammatory action as inhibitors of TNF-α secretion and inhibitors of IL-1 secretion or an analgetic action.

In 1975 TNF-α was defined as a serum factor induced by endotoxin and causing tumour necrosis in vitro and in vivo (Carswell E A et al., Proc. Natl. Acad. Sci. U.S.A., 1975, 72:3666-3670). Besides antitumour action, TNF-α also exhibits numerous other biological actions important in the homeostasis of organisms and in pathophysiological conditions. The main sources of TNF-α are monocytes-macrophages, T-lymphocytes and mastocytes.

The discovery that anti-TNF-a antibodies (cA2) have an action in treating patients with rheumatoid arthritis (RA) (Elliott M et al., Lancet, 1994, 344:1105-1110) led to an increased interest in finding novel TNF-α inhibitors as possible potent drugs for RA. Rheumatoid arthritis is an autoimmune chronic inflammatory disease characterized by irreversible pathological changes in the joints. Besides in RA, TNF-α antagonists may also be used in numerous pathological conditions and diseases such as spondylitis, osteoarthritis, gout and other arthritic conditions, sepsis, septic shock, toxic shock syndrom, atopic dermatitis, contact dermatitis, psoriasis, glomerulonephritis, lupus erythematosus, scleroderma, asthma, cachexia, chronic obstructive lung disease, congestive cardiac arrest, insulin resistance, lung fibrosis, multiple sclerosis, Crohn's disease, ulcerative colitis, viral infections and AIDS.

Evidence for the biological importance of TNF-α was obtained by in vivo experiments in mice, which are deficient in a gen for TNF-α or its receptor. Such animals do not develop a collagen-induced arthritis model (Mori L et al., J. Immunol., 1996, 157:3178-3182) nor an endotoxin-caused septic shock condition (Pfeffer K et al., Cell, 1993, 73:457-467). Animals having an increased TNF-α level fall sick with a chronic inflammatory polyarthritis (Georgopoulos S et al., J. Inflamm., 1996, 46:86-97; Keffer J et al., EMBO J., 1991, 10:4025-4031). The pathological clinical picture of such animals is alleviated by inhibitors of TNF-α production. The treatment of such inflammatory and pathological conditions usually includes the application of non-steroid antiinflammatory drugs and, in more severe cases, gold salts, D-penicillamine or methotrexate are administered. Said drugs act symptomatically, but they do not stop the pathological process. Novel approaches in the therapy of rheumatoid arthritis are based upon drugs such as tenidap, leflunomide, cyclosporin, FK-506 and upon biomolecules neutralizing the TNF-α action. At present there are commercially available ethanercept (Enbrel, Immunex/Wyeth), a fusion protein of the soluble TNF-α receptor, and infliximab (Remicade, Centocor), a chimeric monoclonal human and mouse antibody. Besides in RA therapy, ethanercept and infliximab are also registered for the therapy of Crohn's disease (Exp. Opin. Invest. Drugs, 2000, 9:103).

In RA therapy, besides inhibition of TNF-α secretion, also the inhibition of IL-1 secretion is very important since IL-1 is an important cytokin in cell regulation and immunoregulation as well as in pathophysiological inflammatory conditions (Dinarello C A et al., Rev. Infect. Disease, 1984, 6:51). Well-known biological activities of IL-1 are: the activation of T-cells, the induction of elevated temperature, the stimulation of the secretion of prostaglandine or collagenase, the chemotaxia of neutrophils and the reduction of iron level in plasma (Dinarello C A, J. Clinical Immunology, 1985, 5:287). Two receptors to which IL-1 may bind are well known: IL-1RI and IL-1RII. By binding IL-1, IL-1 RI transfers a signal intracellularly, whereas IL-1RIl, also situated on the cell surface, is not able to do so. Since IL1-RII binds IL-1 as well as IL-1RI, it acts as a negative regulator of IL-1 action. Besides this mechanism of signal transfer regulation, the presence of a natural antagonist of IL-1 receptor (IL-1ra) is proven. This protein binds to IL-1RI but without any possibility of transferring any signal inside the cell. However, IL-1ra has an effect on the breaking of the signal through IL-1RI only if it is present in a concentration 500 times higher than that of IL-1. Recombinant human IL-1ra (Amgen) was clinically tested (Bresnihan B et al., Arthrit. Rheum., 1996, 39:73) and the obtained results indicated an improvement of the clinical picture over a placebo in 472 RA patients. These results indicate the importance of the inhibition of IL-1 action in treating diseases such as RA where IL-1 production is increased. Since there exists a synergistic action of TNF-α and IL-1, novel 1-thia-dibenzoazulene derivatives may be used in treating conditions and diseases related to an enhanced secretion of TNF-α and IL-1.

### INVENTIVE SOLUTION

The present invention relates to novel 1-thia-dibenzoazulene derivatives of the formula I: 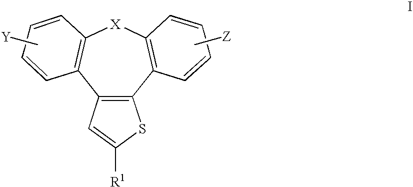
wherein
X individually denotes a hetero atom —O— or —S—;
Y and Z independently from each other individually denote a fragment of the formula II:
   —O—(CH₂)ₘ-A II
   wherein
A individually denotes an optionally substituted C₁-C₇ alkyl or C₂-C₇ alkenyl, C₂-C₇ alkinyl group, an optionally substituted aryl, heteroaryl or heterocyclic, amino, N—(C₁-C₇-alkyl)amino, N,N-di(C₁-C₇-alkyl)amino group;
m denotes an integer from 1 to 4;
R¹ individually denotes hydrogen, halo, an optionally substituted C₁-C₇ alkyl or C₂-C₇ alkenyl, C₂-C₇ alkinyl group, an optionally substituted aryl, heteroaryl or heterocyclic group, hydroxy, hydroxy-C₂-C₇ alkenyl, hydroxy-C₂-C₇ alkinyl, C₁-C₇ alkoxy, thiol, thio-C₂C₇ alkenyl, thio-C₂C₇ alkinyl, C₁-C₇ alkylthiol, amino, N—(C₁-C₇-alkyl)amino, N,N-di(C₁-C₇-alkyl)amino, C₁-C₇ alkylamino, amino-C₂-C₇ alkenyl, amino-C₂-C₇ alkinyl, amino-C₁-C₇ alkoxy, C₁-C₇ alkanoyl, aroyl, oxo-C₁-C₇ alkyl, C₁-C₇ alkanoyloxy, carboxy, an optionally substituted C₁-C₇ alkyloxycarbonyl or aryloxycarbonyl, carbamoyl, N—(C₁-C₇-alkyl)carbamoyl, N,N-di(C₁-C₇-alkyl)carbamoyl, cyano, cyano-C₁-C₇ alkyl, sulfonyl, C₁-C₇ alkylsulfonyl, sulfinyl, C₁-C₇ alkylsulfinyl and nitro group or a subtituent of the formula II, wherein the symbols A and m are defined earlier;
   as well as to pharmacologically acceptable esters, salts and solvates thereof.

The present invention relates also to a process for preparing pharmaceutical formulations containing one or more above-mentioned compounds in an amount effective for the treatment of inflammatory diseases and conditions related to an enhanced secretion of TNF-α and IL-1 in humans and animals.

If not stated otherwise, the following terms have the below meanings.

The term “halo” relates to a halo atom, which may be fluorine, chlorine, bromine or iodine.

The term “alkyl” relates to alkyl groups with the meaning of alkanes, wherefrom radicals are derived, which radicals may be straight, branched or cyclic or a combination of straight and cyclic ones and of branched and cyclic ones. The preferred straight or branched alkyls are e.g. methyl, ethyl, propyl, isopropyl, butyl, sec-butyl and tert-butyl. The preferred cyclic alkyls are e.g. cyclopentyl or cyclohexyl. Alkyl may be optionally additionally substituted with one, two, three or more substituents. Such substituents may be halo atom (preferably fluorine or chlorine), hydroxy, C₁-C₄ alkoxy (preferably methoxy or ethoxy), thiol, C₁-C₄ alkylthio (preferably methylthio or ethylthio), amino, N—(C₁-C₄) alkylamino (preferably N-methylamino or N-ethylamino), N,N-di(C₁-C₄-alkyl)-amino (preferably dimethylamino or diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

The term “alkenyl” relates to alkenyl groups having the meaning of hydrocarbon radicals, which may be straight, branched or cyclic or are a combination of straight and cyclic ones or of branched and cyclic ones, but having at least one carbon-carbon double bond. The most frequent alkenyls are ethenyl, propenyl, butenyl or cyclohexenyl. Alkenyl may be optionally additionally substituted with one, two or three halo atoms. Such substituents may be e.g. 2-chloroethenyl, 1,2-dichloroethenyl or 2-bromo-propen-1-yl.

The term “alkinyl” relates to alkinyl groups having the meaning of hydrocarbon radicals, which are straight or branched and contain at least one and at most two carbon-carbon triple bonds. The most frequent alkinyls are e.g. ethinyl, propinyl or butinyl.

The term “alkoxy” relates to straight or branched chains of alkoxy group. Examples of such groups are methoxy, propoxy, prop-2-oxy, butoxy, but-2-oxy or methylprop-2-oxy.

The term “aryl” relates to groups having the meaning of an aromatic ring, e.g. phenyl, as well as to fused aromatic rings. Aryl contains one ring with at least 6 carbon atoms or two rings with totally 10 carbon atoms and with alternating double (resonant) bonds between carbon atoms. The most freqently used aryls are e.g. phenyl or naphthyl. In general, aryl groups may be linked to the rest of the molecule by any available carbon atom via a direct bond or via a C₁-C₄ alkylene group such as methylene or ethylene. Under the term aryl there is also understood a phenyl ring fused with an optionally substituted cycloalkyl, most frequently with cyclohexane.

The term “heteroaryl” relates to groups having the meaning of aromatic and partially aromatic groups of a monocyclic or bicyclic ring with 4 to 12 carbon atoms, at least one of them being a hetero atom such as O, S or N, and the available nitrogen atom or carbon atom is the binding site of the group to the rest of the molecule. Examples of this type are thiophenyl, pyrrolyl, imidazolyl, pyridinyl, oxazolyl, thiazolyl, pyrazolyl, tetrazolyl, pirimidinyl, pyrazinyl, quinolinyl or triazinyl.

The term “heterocycle” relates to five-member or six-member, fully saturated or partly unsaturated heterocyclic groups containing at least one hetero atom such as O, S or N, and the available nitrogen atom or carbon atom is the binding site of the group to the rest of the molecule. The most frequent examples are morpholinyl, piperidinyl, piperazinyl, pyrrolidinyl, pirazinyl or imidazolyl.

The term “alkanoyl” group relates to straight chains of acyl group such as formyl, acetyl or propanoyl.

The term “aroyl” group relates to aromatic acyl groups such as benzoyl.

Aryl, heteroaryl or heterocycle may be optionally additionally substituted with one, two or more substituents. The substituents may be halo (chlorine or fluorine), C₁-C₄ alkyl (preferably methyl, ethyl or isopropyl), trifluoromethyl, cyano, nitro, hydroxy, C₁-C₄ alkoxy (preferably methoxy or ethoxy), C₁-C₄ alkyloxycarbonyl (preferably methyloxycarbonyl) thiol, C₁-C₄ alkylthio (preferably methylthio or ethylthio), amino, N—(C₁-C₄) alkylamino (preferably N-methylamino or N-ethylamino), N,N-di(C₁-C₄-alkyl)-amino (preferably N,N-dimethylamino or N,N-diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

The term “pharmaceutically suitable salts” relates to salts of the compounds of the formula I (including also quaternary ammonium salts with C₁-C₄ alkylhalides, preferably with methyl bromide and methyl chloride) with inorganic acids (hydrochloric, hydrobromic, phosphoric, metaphosphoric, nitric or sulfuric acids) or with organic acids (tartaric, acetic, citric, maleic, lactic, fumaric, benzoic, succinic, methane sulfonic orp-toluene sulfonic acids).

Some compounds of the formula I may form salts with organic or inorganic acids or bases and these are also included in the present invention.

Solvates (most frequently hydrates) which may be formed by compounds of the formula I or salts thereof are also an object of the present invention.

The present invention also relates to all prodrug forms of the compounds of the formula I, i.e. compounds which in vivo, when administered to mammals, release an active substance of the formula I. Prodrug forms of the compounds of the formula I are prepared by a modification of the functional groups in the compounds of the formula I in such a way that a modified molecule may be cleaved in vivo and release a starting compound. The prodrug forms include the compounds of the formula I, wherein a hydroxy, amino or carboxy group of the compound of the formula I is linked to any group that may be cleaved in vivo and thus regenerate a free hydroxy, amino or carboxy group. Examples of prodrug forms include, but are not limited to esters (e.g. acetate, formiate and benzoate derivatives) of the compounds of the formula I.

Depending upon the nature of particular substituents, the compounds of the formula I may have geometric isomers and one or more chiral centres so that there can exist enantiomers or diastereoisomers. The present invention also relates to such individual isomers and mixtures thereof including racemates. Methods for determining stereochemistry and for the resolution of stereoisomers are well known from the literature.

The present invention also relates to all possible tautomeric forms of particular compounds of the formula I.

In the preparation of the compounds of a specific pharmacological activity represented by the structure I, also certain novel compounds are prepared as intermediates in the preparation of pharmacologically active compounds. The present invention also relates to such intermediates.

“Pharmaceutically acceptable carrier” relates to a carrier which is used in the preparation of a pharmaceutical formulation that is generally safe, non-toxic and biologically acceptable and includes a carrier which is acceptable for pharmaceutical use in humans and animals. “Pharmaceutically acceptable carrier” in the present invention includes one or more such carriers.

A further object of the present invention relates to the preparation of compounds of the formula I according to processes comprising
a) a reaction of condensation of alcohols of the formula IIIa: 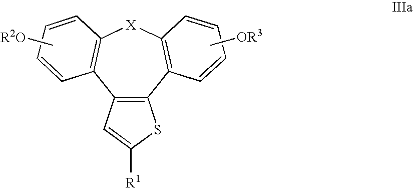
   wherein at least one of the radicals R² and R³ individually denotes hydrogen and the other one a protecting group, whereas R¹ has the meaning as in the formula I;
or a condensation of the compounds of the formula Illb: 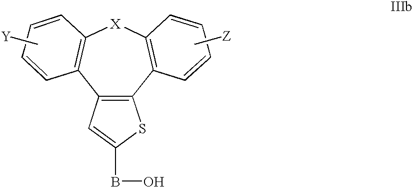
   wherein B individually denotes a —C(O)— or —CH₂— group and Y and Z have the meaning as in the formula I,
with compounds of the formula IVa:
   L¹-(CH₂)ₘ-A IVa
   wherein L¹ has the meaning of a leaving group and m and A have the meaning as previously defined;
b) a reaction of condensation of the compounds of the formula lIlc: 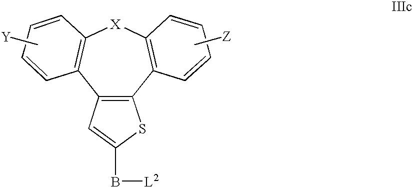
   wherein B individually denotes a —C(O)— or —CH₂— group, L² has the individual meaning of a leaving group and Y and Z have a meaning as in the formula I,
with compounds of the formula IVb:
   HO—(CH₂)ₘ-A IVb
   wherein A and m have the meaning as previously defined.
   Preparation Methods:

a) Compounds of the formula I may be prepared according to the present process by a reaction of compounds of the formula Illa or Illb and of compounds of the formula IVa, wherein L¹ has the meaning of a leaving group, which may be a halo atom (most frequently bromine, chlorine or iodine) or sulfonyloxy group (most frequently trifluoromethylsulfonyloxy or p-toluenesulfonyloxy). The reactions of condensation may be carried out according to methods disclosed for the preparation of analogous compounds (Menozzi G, J. Heterocyclic Chem., 1997, 34:963-968; WO 01/87890; Jones D C et al., J. Med. Chem.; 1984, 27:1057-1066). The reactions are carried out at a temperature from 20° C. to 110° C., preferably 100° C., during 1 to 24 hours in a two-phase system (preferably with 50% NaOH/toluene) in the presence of a phase transfer catalyst (preferably benzyl triethyl ammonium chloride, benzyl triethyl ammonium bromide, cetyl trimethyl bromide) or under heating for several hours in N,N-dimethylformamide in the presence of a base (e.g. potassium carbonate). After treating the reaction mixture, the products formed are isolated by recrystallization or chromatography on a silica gel column.

The starting compounds of the formula Illa or Illb are already known or are prepared according to methods disclosed for the preparation of analogous compounds (WO 01/878990).

Halides of the formula IVa are already known.

b) Compounds of the formula I according to the present process may be prepared by reacting compounds of the formula IIIc, wherein L² has the meaning of a leaving group defined earlier for L¹, and compounds of the formula IVb. The most suitable condensation reactions are reactions of nucleophilic substitution on a saturated and carbonyl carbon atom as disclosed in the literature.

The starting compounds of the formula IlIc (most frequently halides) may be obtained by halogenation (e.g. bromination of chlorination) of the compounds of the formula IIIb with common halogenating agents (hydrobromic acid, PBr₃, SOCl₂ or PCl₅) according to processes disclosed in the literature. The obtained compounds may be isolated or may be used without isolation as suitable intermediates for the preparation of the compounds of the formula I.

Besides the reactions mentioned above, the compounds of the formula I may be prepared by transforming other compounds of the formula I and it is to be understood that the present invention also comprises such compounds and processes.

The formylation of the compounds of the formula I by processes such as e.g. Vilsmeier acylation or the reaction of n-BuLi and N,N-dimethylformamide is a further general example of transformation. The reaction conditions of these processes are well known in the literature.

By hydrolysis of the compounds of the formula I having nitrile, amide or ester groups, there may be prepared compounds with a carboxyl group, which are suitable intermediates for the preparation of other compounds with novel functional groups such as e.g. esters, amides, halides, anhydrides, alcohols or amines.

Oxidation or reduction reactions are a further possibility of the change of substituents in the compounds of the formula I. Most frequently used oxidation agents are peroxides (hydrogen peroxide, m-chloroperbenzoic acid or benzoyl peroxide) or permanganate, chromate or perchlorate ions. Thus e.g. by the oxidation of an alcohol group by pyridinyl dichromate or pyridinyl chlorochromate, an aldehyde group is formed, which group may be converted to a carboxyl group by further oxidation.

By a selective oxidation of an alkylthio group, alkylsulfinyl or alkylsulfonyl groups may be prepared.

By the reduction of compounds with a nitro group, the preparation of amino compounds is made possible. The reaction is carried out under usual conditions of catalytic hydrogenation or electrochemically. By catalytic hydrogenation using palladium on carbon, alkenyl substituents may be converted to alkyl ones or nitrile group can be converted to alkylamine.

Various substituents of the aromatic structure in the compounds of the formula I may be introduced by standard substitution reactions or by usual changes of individual functional groups. Examples of such reactions are aromatic substitutions, alkylations, halogenation, hydroxylation as well as oxidation or reduction of substituents. Reagents and reaction conditions are known from the literature. Thus e.g. by aromatic substitution a nitro group is introduced in the presence of concentrated nitric acid and sulfuric acid. By using acyl halides or alkyl halides, the introduction of acyl groups or alkyl groups is made possible. Such reactions are carried out in the presence of Lewis acids such as aluminum- or iron-trichloride in conditions of Friedel-Crafts reaction. By the reduction of the nitro group, an amino group is obtained, which is by the reaction of diazotizing converted to a suitable starting group, which may be replaced with one of the following groups: H, CN, OH or halo. The reduction of a nitro group of aromatic compounds may be carried out in the presence of reduction agents such as Zn, Sn or FE, or by catalytic hydrogenation.

In order to prevent undesired interaction in chemical reactions, it is often necessary to protect certain groups such as e.g. hydroxy, amino, thio or carboxy. For this purpose a great number of protecting groups may be used (Green TW, Wuts PGH, Protective Groups in Organic Synthesis, John Wiley and Sons, 1999) and the choice, use and elimination thereof are conventional methods in chemical synthesis.

A convenient protection for amino or alkylamino groups are groups such as e.g. alkanoyl (acetyl), alkoxycarbonyl (methoxycarbonyl, ethoxycarbonyl or tert-butoxycarbonyl); arylalkyloxycarbonyl (benzyloxycarbonyl), aroyl (benzoyl) or alkylsilyl (trimethylsilyl or trimethylsilylethoxymethyl) groups. The conditions of removing a protecting group depend upon the choice and the characteristics of this group. Thus e.g. acyl groups such as alkanoyl, alkoxycarbonyl or aroyl may be eliminated by hydrolysis in the presence of a base (sodium hydroxide or potassium hydroxide), tert-butoxycarbonyl or alkylsilyl (trimethylsilyl) may be eliminated by treatment with a suitable acid (hydrochloric, sulfuric, phosphoric or trifluoroacetic acid), whereas arylmethoxycarbonyl group (benzyloxycarbonyl) may be eliminated by hydrogenation using a catalyst such as palladium on carbon.

The most frequent protection for a hydroxy group is acetyl, benzoyl or benzyl.

Salts of the compounds of the formula I may be prepared by generally known processes such as e.g. by reacting the compounds of the formula I with a corresponding base or acid in an appropriate solvent or solvent mixture e.g. ethers (diethylether) or alcohols (ethanol, propanol or isopropanol).

Another object of the present invention concerns the use of the present compounds in the therapy of inflammatory diseases and conditions, especially all diseases and conditions induced by excessive TNF-α and IL-1 secretion.

The inhibitors of production of cytokins or inflammation mediators, which are the object of the present invention, or pharmacologically acceptable salts thereof may be used in the production of drugs for the treatment and prophylaxis of any pathological condition or disease induced by excessive unregulated production of cytokins or inflammation mediators, which drugs should contain an effective dose of said inhibitors.

The present invention specifically relates to an effective dose of TNF-α inhibitor, which may be determined by usual methods.

Further, the present invention relates to a pharmaceutical formulation containing an effective non-toxic dosis of the present compounds as well as pharmaceutically acceptable carriers or solvents.

The preparation of pharmaceutical formulations may include blending, granulating, tabletting and dissolving the ingredients. Pharmaceutically acceptable carriers (binders and fillers) may be solid or liquid. Solid carriers may be lactose, sucrose, talcum, gelatine, agar, pectin, magnesium stearate, fatty acids etc. Liquid carriers may be syrups, oils such as olive oil, sunflower oil or soya bean oil, water etc. Similarly, the pharmaceutically acceptable formulations may also contain a component for a sustained release of the active component such as e.g. glyceryl monostearate or glyceryl distearate. Various forms of pharmaceutical formulations may be used. Thus, if a solid carrier is used, these forms may be tablets, hard gelatine capsules, powder or granules, which may be administered in capsules per os. The amount of the solid carrier may vary, but it is mainly from 25 mg to 1 g. If a liquid carrier is used, the formulation would be in the form of a syrup, emulsion, soft gelatine capsules, sterile injectable liquids such as ampoules or non-aqueous liquid suspensions.

The compounds of the present invention may be applied per os, parenterally, locally, intranasally, intrarectally and intravaginally. The parenteral route herein means intravenous, intramuscular and subcutaneous applications. Appropriate formulations of the present compounds may be used in the prophylaxis as well as in the treatment of various diseases and conditions induced by an excessive unregulated production of cytokins or inflammation mediators, primarily TNF-α. They comprise e.g. rheumatoid arthritis, rheumatoid spondylitis, osteoarthritis and other arthritic pathological conditions and diseases, eczemas, psoriasis and other inflammatory skin conditions such as burns caused by UV radiation (sun rays and similar UV sources), inflammatory eye diseases, Crohn's disease, ulcerative colitis and asthma.

The inhibitory action of the present compounds upon TNF-α and IL-1 secretion was determined by the following in vitro and in vivo experiments:

Determination of TNF-α and IL-1 Secretion in Human Peripheral Blood Mononuclear Cells in vitro

Human peripheral blood mononuclear cells (PBMC) were prepared from heparinized whole blood after separating PBMC on Ficoll-Paque™Plus (Amersham-Pharmacia). To determine the TNF-α level, 3.5-5×10⁴ cells were cultivated in a total volume of 200 μl for 18 to 24 hours on microtitre plates with a flat bottom (96 wells, Falcon) in RPMI 1640 medium, into which there were added 10% FBS (Fetal Bovine Serum, Biowhittaker) previously inactivated at 54° C./30 min, 100 units/ml of penicillin, 100 mg/ml of streptomycin and 20 mM HEPES (GIBCO). The cells were incubated at 37° C. in an atmosphere with 5% CO₂ and 90% humidity. In a negative control the cells were cultivated only in the medium (NC), whereas in a positive control TNF-α secretion was triggered by adding 1 ng/ml of lipopolysaccharides (LPS, E. coli serotype 0111:B4, SIGMA) (PC). The effect of the tested substances upon TNF-α secretion was investigated after adding them into cultures of cells stimulated by LPS (TS). The TNF-α level in the cell supernatant was determined by ELISA procedure according to the suggestions of the producer (R&D Systems). The test sensitivity was <3 pg/ml TNF-α. The IL-1 level was determined in an assay under the same conditions and with the same number of cells and the same concentration of the stimulus by ELISA procedure (R&D Systems). The percentage of inhibition of TNF-α or IL-1 production was calculated by the equation:
% inhibition=[1−(TS−NC)/(PC−NC)]*100.

The IC₅₀ value was defined as the substance concentration, at which 50% of TNF-α production were inhibited.

Compounds showing IC₅₀ with 20 μM or lower concentrations are active.

Determination of TNF-α and IL-1 Secretion in Mouse Peritoneal Macrophages in vitro

In order to obtain peritoneal macrophages, Balb/C mouse strain males, age 8 to 12 weeks, were injected i.p. with 300 μg of zymosan (SIGMA) dissolved in a phosphate buffer (PBS) in a total volume of 0.1 ml/mouse. After 24 hours the mice were euthanized according to the Laboratory Animal Welfare Act. The peritoneal cavity was washed with a sterile physiological solution (5 ml). The obtained peritoneal macrophages were washed twice with a sterile physiological solution and, after the last centrifugation (350 g/10 min), resuspended in RPMI 1640, into which 10% of FBS were added. In order to determine TNF-α secretion, 5×10⁴ cells/well were cultivated in a total volume of 200 μl for 18 to 24 hours on microtitre plates with a flat bottom (96 wells, Falcon) in RPMI 1640 medium, into which 10% FBS (Fetal Bovine Serum, Biowhittaker) inactivated by heat, 100 units/ml of penicillin, 100 mg/ml of streptomycin, 20 mM HEPES and 50 μM 2-mercaptoethanol (all of GIBCO) were added. The cells were incubated at 37° C. in an atmosphere with 5% CO₂ and 90% humidity. In a negative control the cells were cultivated only in a medium (NC), whereas in a positive control the TNF-α secretion was triggered by adding 10 ng/ml of lipopolysaccharides (LPS, E. coli serotype 0111:B4, SIGMA) (PC). The effect of the substances upon the TNF-α secretion was investigated after adding them into cultures of cells stimulated with LPS (TS). The TNF-α level in the cell supernatant was determined by ELISA procedure (R&D Systems, Biosource). IL-1 level was determined in an assay, identical to an assay for TNF-a by ELISA procedure (R&D Systems). The percentage of inhibition of TNF-α or IL-1 production was calculated by the equation:
% inhibition=[1−(TS−NC)/(PC−NC)]*100.

The IC₅₀ value was defined as the substance concentration, at which 50% of TNF-α production were inhibited.

Compounds showing IC₅₀ with 10 μM or lower concentrations are active.

In vivo Model of LPS-Induced Excessive TNF-α or IL-1 Secretion in Mice

TNF-α or IL-1 secretion in mice was induced according to the already disclosed method (Badger A M et al., J. Pharmac. Env. Therap., 1996, 279:1453-1461). Balb/C males, age 8 to 12 weeks, in groups of 6 to 10 animals were used. The animals were treated p.o. either with a solvent only (in negative and in positive controls) or with solutions of substances 30 minutes prior to i.p. treatment with LPS (E. coli serotype 0111:B4, Sigma) in a dosis of 25 μg/animal. Two hours later the animals were euthanized by means of an i.p. Roumpun (Bayer) and Ketanest (Parke-Davis) injection. A blood sample of each animal was taken into a Vacutainer tube (Becton Dickinson) and the plasma was separated according to the producer's instructions. The TNF-α level in the plasma was determined by ELISA procedure (Biosource, R&D Systems) according to the producer's instructions. The test sensitivity was <3 pg/ml TNF-α. The IL-1 level was determined by ELISA procedure (R&D Systems). The percentage of inhibition of TNF-α or IL-1 production was calculated by the equation:
% inhibition=[1−(TS−NC)/(PC−NC)]*100.

Active are the compounds showing a 30% or more inhibition of TNF-α production at a dosis of 10 mg/kg.

Writhing Assay for Analgetic Activity

In this assay pain is induced by the injection of an irritant, most frequently acetic acid, into the peritoneal cavity of mice. Animals react with characteristic writhings, which has given the name to the assay (Collier H O J et al., Pharmac. Chemother., 1968, 32:295-310; Fukawa K et al., J. Pharmacol. Meth., 1980, 4:251-259; Schweizer A et al., Agents Actions, 1988, 23:29-31). The assay is convenient for the determination of analgetic activity of compounds. Procedure: male Balb/C mice (Charles River, Italy), age 8 to 12 weeks, were used. A control group received methyl cellulose p.o. 30 minutes prior to i.p. application of acetic acid in a concentration of 0.6%, whereas test groups received standard (acetylsalicylic acid) or test substances in methyl cellulose p.o. 30 minutes prior to i.p. application of 0.6% acetic acid (volume 0.1 ml/10 g). The mice were placed individually under glass funnels and the number of writhings was registered for 20 minutes for each animal. The percentage of writhing inhibition was calculated according to the equation:
% inhibition=(mean value of number of writhings in the control group−number of writhings in the test group)/number of writhings in the control group*100.

Active are the compounds showing such analgetic activity as acetylsalicylic acid or better.

In vivo Model of LPS-Induced Shock in Mice

Male Balb/C mice (Charles River, Italy), age 8 to 12 weeks, were used. LPS isolated from Serratia marcescens (Sigma, L-6136) was diluted in sterile physiological solution. The first LPS injection was administered intradermally in a dosis of 4 μg/mouse. 18 to 24 hours later, LPS was administered i.v. in a dosis of 200 μg/mouse. A control group received two LPS injections as disclosed above. The test groups received substances p.o. half an hour prior to each LPS application. The survival after 24 hours was observed.

Active are the substances at which the survival at a dosis of 30 mg/kg was 40% or more.

Compounds from Examples 4 and 14 show activity in at least two investigated assays though these results only represent an illustration of the biological activity of the compounds and should not limit the invention in any way.

### PREPARATION PROCESSES WITH EXAMPLES

The present invention is ilustrated by the following Examples, which in no way represent a limitation thereof.

### Example 1

### 10-Benzyloxy-8-oxa-1-thia-dibenzo[e,h]azulene-2-carboxylic acid ethyl ester

To a solution of 10-hydroxy-8-oxa-1-thia-dibenzo[e,h]azulene-2-carboxylic acid ethyl ester (3) (0.30 mmol) in N,N-dimethylformamide (2.0 mL), potassium carbonate (0.74 mmol) was added. The reaction mixture was heated to 100° C., benzyl chloride (0.36 mmol) was added and then the heating of the reaction mixture at 100° C. was continued for another 2 hours. After 2 hours the reaction mixture was cooled to room temperature and filtered. The filtrate was evaporated under reduced pressure. Water was added to the evaporation residue and it was extracted with ethyl acetate, dried over anhydrous Na₂SO₄ and evaporated under reduced pressure. After purifying by chromatography over a silicagel column a crystalline product was isolated.

¹H NMR (ppm, CDCl₃): 8.01 (s, 1H), 7.52-6.82 (m, 12H), 5.09 (s, 2H), 4.39 (q, 2H), 1.42 (t, 3H);

MS (m/z): 429.4 [MH⁺].

### Example 2

### 10-(3-Dimethylamino-propoxy)-8-oxa-1-thia-dibenzo[e,h]azulene-2-carboxylic acid ethyl ester

According to the process disclosed in Example 1, from 10-hydroxy-8-oxa-1-thia-dibenzo[e,h]azulene-2-carboxylic acid ethyl ester (3) (0.30 mmol) and 3-dimethylaminopropylchloride hydrochloride (0.36 mmol) an oily product was obtained.

¹H NMR (ppm, CDCl₃): 8.01 (s, 1H), 7.51-6.74 (m, 7H), 4.39 (q, 2H), 4.07 (t, 2H), 2.59 (t, 2H), 2.37 (s, 6H), 2.06 (m, 2H), 1.42 (t, 3H);

MS (m/z): 424.3 [MH⁺].

### Example 3

### 10-(2-Dimethylamino-ethoxy)-8-oxa-1-thia-dibenzo[e,h]azulene-2-carboxylic acid ethyl ester

According to the process disclosed in Example 1, from 10-hydroxy-8-oxa-1-thia-dibenzo[e,h]azulene-2-carboxylic acid ethyl ester (3) (0.30 mmol) and 2-dimethylaminoethylchloride hydrochloride (0.36 mmol) an oily product was obtained.

¹H NMR (ppm, CDCl₃): 8.07 (s, 1H), 7.51-6.77 (m, 7H), 4.39 (q, 2H), 4.19 (t, 2H), 2.88 (t, 2H), 2.47 (s, 6H), 1.42 (t, 3H);

MS (m/z): 410.3 [MH⁺].

### Example 4

### 10-(2-Pyrrolidin-1-yl-ethoxy)-8-oxa-1-thia-dibenzo[e,h]azulene-2-carboxylic acid ethyl ester

According to the process disclosed in Example 1, from 10-hydroxy-8-oxa-1-thia-dibenzo[e,h]azulene-2-carboxylic acid ethyl ester (3) (0.30 mmol) and 1-(2-chloro-ethyl)-pyrrolidine hydrochloride (0.36 mmol) an oily product was obtained.

¹H NMR (ppm, CDCl₃): 7.97 (s, 1H), 7.47-6.73 (m, 7H), 4.35 (q, 2H), 4.16 (t, 2H), 2.95 (t, 2H), 2.69 (bs, 4H), 1.37 (t, 3H), 1.82 (bs, 4H);

MS (m/z): 436.3 [MH⁺].

### Example 5

### 11-Benzyloxy-8-oxa-1-thia-dibenzo[e,h]azulene-2-carboxylic acid ethyl ester

According to the process disclosed in Example 1, from 11-hydroxy-8-oxa-1-thia-dibenzo[e,h]azulene-2-carboxylic acid ethyl ester (4) (0.59 mmol) and benzyl chloride (0.71 mmol) an oily product was obtained.

¹H NMR (ppm, CDCl₃): 8.04 (s, 1H), 7.54-6, 98 (m, 12H), 5.08 (s, 2H), 4.43 (m, 2H), 1.45 (t, 3H).

### Example 6

### 11-(3-Dimethylamino-propoxy)-8-oxa-1-thia-dibenzo[e,h]azulene-2-carboxylic acid ethyl ester

According to the process disclosed in Example 1, from 11-hydroxy-8-oxa-1-thia-dibenzo[e,h]azulene-2-carboxylic acid ethyl ester (4) (0.74 mmol) and 3-dimethylaminopropylchloride hydrochloride (0.89 mmol) an oily product was obtained.

¹H NMR (ppm, CDCl₃): 8.02 (s, 1H), 7.52-6.89 (m, 7H), 4.41 (q, 2H), 4.02 (t, 2H), 2.58 (t, 2H), 2.36 (s, 6H), 2.03 (m, 2H), 1.42 (t, 3H);

MS (m/z): 424.3 [MH⁺].

### Example 7

### 10-(3-Dimethylamino-propoxy)-8-oxa-1-thia-dibenzo[e,h]azulene-2-carboxylic acid

To a solution of 10-(3-dimethylamino-propoxy)-8-oxa-1-thia-dibenzo[e,h]azulene-2-carboxylic acid ethyl ester (0.10 mmol) in ethanol (2.0 mL) a 1M sodium hydroxide solution (1.0 mL) was added. The reaction mixture was stirred at 80° C. for 2 hours, then it was cooled to room temperature, diluted with water and extracted with ethyl acetate. The aqueous extract was acidified with concentrated hydrochloric acid to pH 5-6 and a white crystalline product was precipitated and filtered off.

MS (m/z): 396.2 [MH^{+].}

### Example 8

### (10-Benzyloxy-8-oxa-1-thia-dibenzo[e,h]azulene-2-yl)-methanol

To a suspension of lithium aluminum hydride (0.92 mmol) in dry diethyl ether (5.0 mL) a solution of 10-benzyloxy-8-oxa-1-thia-dibenzo[e,h]azulene-2-carboxylic acid ethyl ester (0.46 mmol) in dry diethyl ether (5.0 mL) was added drop by drop. The reaction mixture was stirred for 2 hours at room temperature, then the excess of lithium aluminum hydride was decomposed by adding diethyl ether and water. The obtained white precipitate was filtered off and the filtrate was evaporated under reduced pressure after drying over anhydrous Na₂SO₄. After purifying the evaporation residue by chromatography on a silica gel column, a crystalline product was isolated.

¹H NMR (ppm, CDCl₃): 7.45-6.79 (m, 13H), 5.08 (s, 2H), 4.89 (s, 2H);

MS (m/z): 409.2 [MNa⁺].

### Example 9

### (11-Benzyloxy-8-oxa-1-thia-dibenzo[e,h]azulene-2-yl)-methanol

According to the process disclosed in Example 8, from 11-benzyloxy-8-oxa-1-thia-dibenzo[e,h]azulene-2-carboxylic acid ethyl ester (0.41 mmol) a crystalline product was obtained.

¹H NMR (ppm, CDCl₃): 7.46-6.89 (m, 13H), 5.05 (s, 2H), 4.89 (s, 2H);

MS (m/z): 384.8[M⁻].

### Example 10

### [10-(3-Dimethylamino-propoxy)-8-oxa-1-thia-dibenzo[e,h]azulene-2-yl]-methanol

According to the process disclosed in Example 8, from 10-(3-dimethylamino-propoxy)-8-oxa-1-thia-dibenzo[e,h]azulene-2-carboxylic acid ethyl ester (0.65 mmol) an oily product was obtained.

¹H NMR (ppm, CDCl₃): 7.45-6.67 (m, 8H), 4.88 (s, 2H), 4.01 (t, 2H), 2.58 (t, 2H), 2.36 (s, 6H), 2.03 (m, 2H);

MS (m/z): 382.4 [MH⁺].

### Example 11

### [11-(3-Dimethylamino-propoxy)-8-oxa-1-thia-dibenzo[e,h]azulene-2-yl]-methanol

According to the process disclosed in Example 8, from 11-(3-dimethylamino-propoxy)-8-oxa-1-thia-dibenzo[e,h]azulene-2-carboxylic acid ethyl ester (0.44 mmol) an oily product was obtained.

¹H NMR (ppm, CDCl₃): 7.46-6.76 (m, 8H), 4.89 (s, 2H), 3.97 (t, 2H), 2.55 (t, 2H), 2.34 (s, 6H), 1.99 (m, 2H);

MS (m/z): 382.1 [MH⁺].

### Example 12

### [3-(10-Benzyloxy-8-oxa-1-thia-dibenzo[e,h]azulene-2-ylmethoxy)-propyl]-dimethyl-amine

To a solution of 3-dimethylaminopropylchloride hydrochloride (3.34 mmol) in 50% sodium hydroxide (3.0 mL), a catalytic amount of benzyltriethylammonium chloride and a solution of (10-benzyloxy-8-oxa-1-thia-dibenzo[e,h]azulene-2-yl)-methanol (0.24 mmol) in toluene (7.7 mL) were added. The reaction mixture was heated for 3 hours under vigorous stirring and reflux, then cooled to room temperature, diluted with water and extracted with dichloromethane. The organic extract was washed with water, dried over anhydrous Na₂SO₄ and evaporated under reduced pressure. After purifying by chromatography on a silica gel column an oily product was isolated.

¹H NMR (ppm, CDCl₃): 7.46-6.79 (m, 13H), 5.07 (s, 2H), 4.69 (s, 2H), 3.60 (t, 2H), 2.42 (t, 2H), 2.26 (s, 6H), 1.83 (m, 2H);

MS (m/z): 472.3 [MH⁺].

### Example 13

### [3-(11-Benzyloxy-8-oxa-1-thia-dibenzo[e,h]azulene-2-ylmethoxy)-propyl]-dimethyl-amine

According to the process disclosed in Example 12, from (11-benzyloxy-8-oxa-1-thia-dibenzo[e,h]azulene-2-yl)-methanol (0.26 mmol) and 3-dimethylaminopropylchloride hydrochloride (3.63 mmol) an oily product was obtained.

¹H NMR (ppm, CDCl₃): 7.47-6.89 (m, 13H), 5.05 (s, 2H), 4.72 (s, 2H), 3.64 (t, 2H), 2.59 (t, 2H), 2.39 (s, 6H), 1.93 (m, 2H);

MS (m/z): 472.2 [MH⁺].

### Example 14

### {3-[2-(3-Dimethylamino-propoxymethyl)-8-oxa-1-thia-dibenzo[e,h]azulene-10-yloxy]-propyl}-dimethyl-amine

According to the process disclosed in Example 12, from [10-(3-dimethylamino-propoxy)-8-oxa-1-thia-dibenzo[e,h]azulene-2-yl]-methanol (0.34 mmol) and 3-dimethylaminopropylchloride hydrochloride (4.77 mmol) a crystalline product was obtained.

¹H NMR (ppm, CDCl₃): 7.46-6.72 (m, 8H), 4.70 (s, 2H), 4.04 (t, 2H), 3.62 (t, 2H), 2.51-2.47 (m, 4H), 2.32 (s, 6H), 2.29 (s, 6H), 1.99 (m, 2H), 1.88 (m, 2H);

MS (m/z): 467.4 [MH⁺].

### Example 15

### {3-[2-(3-Dimethylamino-propoxymethyl)-8-oxa-1-thia-dibenzo[e,h]azulene-11-yloxy]-propyl}-dimethyl-amine

According to the process disclosed in Example 12, from [11-(3-dimethylamino-propoxy)-8-oxa-1-thia-dibenzo[e,h]azulene-2-yl]-methanol (0.39 mmol) and 3-dimethylaminopropylchloride hydrochloride (5.52 mmol) an oily product was obtained.

MS (m/z): 467.4 [MH⁺].

### Example 16

### 10-Methoxy-8-oxa-1-thia-dibenzo[e,h]azulene-2-carboxylic acid 2-dimethylamino-ethyl ester

To a solution of 10-methoxy-8-oxa-1-thia-dibenzo[e,h]azulene-2-carboxylic acid (5) (0.31 mmol) in N,N-dimethylformamide (2.0 mL), potassium carbonate (0.75 mmol) was added. The reaction mixture was heated to 100° C., 2-dimethylaminoethylchloride hydrochloride (0.37 mmol) was added and then the heating of the reaction mixture at 100° C. was continued for another 2 hours. After 2 hours the reaction mixture was cooled to room temperature and filtered. The filtrate was evaporated under reduced pressure. To the evaporation residue water was added, it was extracted with ethyl acetate, dried over anhydrous Na₂SO₄ and evaporated under reduced pressure. After purifying by chromatography on a silica gel column an oily product was isolated.

¹H NMR (ppm, CDCl₃): 8.04 (s, 1H), 7.51-6.77 (m, 7H), 4.52 (t, 2H), 3.85 (s, 3H), 2.86 (bs, 2H), 2.46 (s, 6H);

MS (m/z): 396.4 [MH⁺].

### Example 17

### 10-Methoxy-8-oxa-1-thia-dibenzo[e,h]azulene-2-carboxylic acid 3-dimethylamino-propyl ester

According to the process disclosed in Example 16, from 10-methoxy-8-oxa-1-thia-dibenzo[e,h]azulene-2-carboxylic acid (5) (0.31 mmol) and 3-dimethylaminopropylchloride hydrochloride (0.37 mmol) an oily product was obtained.

¹H NMR (ppm, CDCl₃): 8.01 (s, 1H), 7.51-6.77 (m, 7H), 4.42 (t, 2H), 3.85 (s, 3H), 2.66 (bs, 2H), 2.45 (s, 6H), 2.10 (bs, 2H);

MS (m/z): 410.4 [MH⁺].

### Example 18

### 11-Chloro-8-oxa-1-thia-dibenzo[e,h]azulene-2-carboxylic acid 2-dimethylamino-ethyl ester

According to the process disclosed in Example 16, from 11-chloro-8-oxa-1-thia-dibenzo[e,h]azulene-2-carboxylic acid (0.31 mmol) and 2-dimethylaminoethylchloride hydrochloride (0.37 mmol) a crystalline product was obtained.

¹H NMR (ppm, CDCl₃): 8.06 (s, 1H), 7.53-7.22 (m, 7H), 4.52 (t, 2H), 2.84 (t, 2H), 2.44 (s, 6H);

MS (m/z): 400.3 [MH⁺].

### Example 19

### 11-Chloro-8-oxa-1-thia-dibenzo[e,h]azulene-2-carboxylic acid 3-dimethylamino-propyl ester

According to the process disclosed in Example 16, from 11-chloro-8-oxa-1-thia-dibenzo[e,h]azulene-2-carboxylic acid (0.31 mmol) and 3-dimethylaminopropylchloride hydrochloride (0.37 mmol) a crystalline product was obtained.

¹H NMR (ppm, CDCl₃): 8.03 (s, 1H), 7.53-7.19 (m, 7H), 4.42 (t, 2H), 2.55 (t, 2H), 2.36 (s, 6H), 2.03 (m, 2H);

MS (m/z): 414.3 [MH⁺].

### Example 20

### 10-Hydroxy-8-oxa-1-thia-dibenzo[e,h]azulene-2-carboxylic acid 3-dimethylamino-propyl ester

### 10-(3-dimethylamino-propoxy)-8-oxa-1-thia-dibenzo[e,h]azulene-2-carboxylic acid 3-dimethylamino-propyl ester

According to the process disclosed in Example 16, from 10-hydroxy-8-oxa-1-thia-dibenzo[e,h]azulene-2-carboxylic acid (7) (0.19 mmol) and 3-dimethylaminopropylchloride hydrochloride (0.23 mmol) there were obtained 10-hydroxy-8-oxa-1-thia-dibenzo[e,h]azulene-2-carboxylic acid 3-dimethylamino-propyl ester;

MS (m/z): 396.0 [MH⁺];

and 10-(3-dimethylamino-propoxy)-8-oxa-1-thia-dibenzo[e,h]azulene-2-carboxylic acid 3-dimethylamino-propyl ester;

MS (m/z): 481.4 [MH⁺].

### Example 21

### Dimethyl-[3-(8-oxa-1-thia-dibenzo[e,h]azulene-10-yloxy)-propyl]-amine

To a solution of 8-oxa-1-thia-dibenzo[e,h]azulene-10-ol (11) (0.11 mmol) in N,N-dimethylformamide (0.7 mL), potassium carbonate (0.28 mmol) was added. The reaction mixture was heated to 100° C., 3-dimethylaminopropylchloride hydrochloride (0.14 mmol) was added and then the heating of the reaction mixture at 100° C. was continued for another 2 hours. After 2 hours the reaction mixture was cooled to room temperature and filtered. The filtrate was evaporated under reduced pressure. To the evaporation residue water was added and it was extracted with ethyl acetate, dried over anhydrous Na₂SO₄ and evaporated under reduced pressure. After purifying by chromatography on a silica gel column an oily product was isolated.

MS (m/z): 352.3 [MH⁺].

### PREPARATION OF STARTING COMPOUNDS AND INTERMEDIATES

### Method A

### 10-Hydroxy-8-oxa-1-thia-dibenzo[e,h]azulene-2-carboxylic acid ethyl ester (3)

To a solution of 10-methoxy-8-oxa-1-thia-dibenzo[e,h]azulene-2-carboxylic acid ethyl ester (1) (0.43 mmol) in dichloromethane (3.40 mL), a 0.1 M solution of boron(III) bromide in dichloromethane (2.56 mL) was added under stirring at 0° C. The reaction mixture was stirred for 4 hours at room temperature, then water was added and it was extracted with dichloromethane. The organic extract was dried over anhydrous Na₂SO₄ and evaporated under reduced pressure. After purifying by chromatography on a silica gel column a crystalline product was isolated.

By an analogous process starting from 11-methoxy-8-oxa-1-thia-dibenzo[e,h]azulene-2-carboxylic acid ethyl ester (2), 11-hydroxy-8-oxa-1-thia-dibenzo[e,h]azulene-2-carboxylic acid ethyl ester (4) was prepared.

### Method B

### 10-Methoxy-8-oxa-1-thia-dibenzo[e,h]azulene-2-carboxylic acid (5)

To a solution of 10-methoxy-8-oxa-1-thia-dibenzo[e,h]azulene-2-carboxylic acid ethyl ester (1) (1.37 mmol) in ethanol (10.0 mL), a 1M sodium hydroxide solution (11.2 mL) was added. The reaction mixture was stirred at 80° C. for 2 hours, then it was cooled to room temperature, diluted with water and extracted with ethyl acetate. The aqueous extract was acidified with concentrated hydrochloric acid to pH 5-6 and a white crystalline product was precipitated, which was filtered off.

By an analogous process starting from:

11-methoxy-8-oxa-1-thia-dibenzo[e,h]azulene-2-carboxylic acid ethyl ester (2);

10-hydroxy-8-oxa-1-thia-dibenzo[e,h]azulene-2-carboxylic acid ethyl ester (3);

11-hydroxy-8-oxa-1-thia-dibenzo[e,h]azulene-2-carboxylic acid ethyl ester (4);

there were respectively prepared:

11-methoxy-8-oxa-1-thia-dibenzo[e,h]azulene-2-carboxylic acid (6);

10-hydroxy-8-oxa-1-thia-dibenzo[e,h]azulene-2-carboxylic acid (7);

11-hydroxy-8-oxa-1-thia-dibenzo[e,h]azulene-2-carboxylic acid (8).

### Method C

### 10-Methoxy-8-oxa-1-thia-dibenzo[e,h]azulene (9)

A finely ground mixture of 10-methoxy-8-oxa-1-thia-dibenzo[e,h]azulene-2-carboxylic acid (5) (0.31 mmol) and elementary copper (75.0 mg) was heated at 230° C. for about 2 hours. The reaction mixture was cooled to room temperature, dissolved in ethyl acetate and washed with water. The organic extract was dried over anhydrous Na₂SO₄ and evaporated under reduced pressure. After purifying by chromatography on a silica gel column a crystalline product was isolated.

By an analogous process starting from:

11-methoxy-8-oxa-1-thia-dibenzo[e,h]azulene-2-carboxylic acid (6);

10-hydroxy-8-oxa-1-thia-dibenzo[e,h]azulene-2-carboxylic acid (7);

11-hydroxy-8-oxa-1-thia-dibenzo[e,h]azulene-2-carboxylic acid (8);

there were respectively prepared:

11-methoxy-8-oxa-1-thia-dibenzo[e,h]azulene (10);

10-hydroxy-8-oxa-1-thia-dibenzo[e,h]azulene (11);

11-hydroxy-8-oxa-1-thia-dibenzo[e,h]azulene (12).

TABLE 1
Characteristics of the prepared starting compounds and intermediates:

| Cpd. | X | Y | Z | R¹ | MS (m/z) | ¹H NMR (ppm) |
|---|---|---|---|---|---|---|
| 1 | O | H | 10-OCH₃ | CO₂Et | 375.2 (MNa⁺) | 8.08(s, 1H), 7.51-6.75(m, 7H), 4.40(q, 2H), 3.84(s, 3H), 1.42(t, 3H)(CDCl₃) |
| 2 | O | H | 11-OCH₃ | CO₂Et | 353.2 (MH⁺) | 8.02(s, 1H), 7.52-6.89(m, 7H), 4.41(q, 2H), 3.81(s, 3H), 1.42(t, 3H)(CDCl₃) |
| 3 | O | H | 10-OH | CO₂Et | 361.2 (MNa⁺) | 8.01(s, 1H), 7.60-6.69(m, 7H), 4.40(q, 2H), 1.42(t, 3H)(CDCl₃) |
| 4 | O | H | 11-OH | CO₂Et | 361.0 (MNa⁺) | 8.02(s, 1H), 7.51-6.83(m, 7H), 4.41(q, 2H), 1.42(t, 3H)(CDCl₃) |
| 5 | O | H | 10-OCH₃ | CO₂H | 325.0 (MH⁺) | 13.00(bs, 1H), 8.07(s, 1H), 7.95-6.89(m, 7H), 3.83(s, 3H)(DMSO-d₆) |
| 6 | O | H | 11-OCH₃ | CO₂H | 325.0 (MH⁺) | — |
| 7 | O | H | 10-OH | CO₂H | 311.0 (MH⁺) | 12.00(bs, 1H), 10.27(s, 1H), 8.04(s, 1H), 7.68-6.74(m, 7H)(DMSO-d₆) |
| 8 | O | H | 11-OH | CO₂H | 311.0 (MH⁺) | — |
| 9 | O | H | 10-OCH₃ | H | 281.1 (MH⁺) | 7.53-6.47(m, 9H), 3.84(s, 3H)(CDCl₃) |
| 10 | O | H | 11-OCH₃ | H | 281.1 (MH⁺) | — |
| 11 | O | H | 10-OH | H | 266.9 (MH⁺) | — |
| 12 | O | H | 11-OH | H | 266.9 (MH⁺) | — |

1. Compound of the formula I 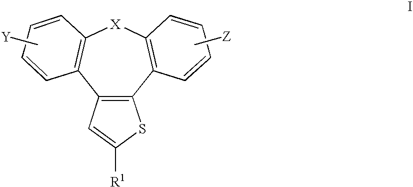
wherein
X denotes a hetero atom —O— or —S—;
Y is H;
Z is a fragment of the formula II
—O—(CH₂)ₘ-A II
wherein
A denotes hydrogen, an C₁-C₇ alkyl which is unsubstituted or substituted by 1 to 3 groups selected from halo, hydroxy, C₁-C₄ alkoxy, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; C₂-C₇ alkenylwhich is unsubstituted or substituted by 1 to 3 halo atoms; C₂-C₇ alkinyl; monocyclic or bicyclic aryl group having from 6 to 10 carbon atoms and alternating double bonds between carbon atoms, wherein the monocyclic or bicyclic aryl group is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; C₄-C₁₂ monocyclic or bicyclic heteroaryl ring system which is aromatic or partially aromatic containing at least one heteroatom selected from oxygen, sulphur and nitrogen, wherein C₄-C₁₂ monocyclic or bicyclic heteroaryl ring system is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; 5-6 membered heterocyclic group containing at least 1 heteroatom selected from oxygen, sulphur and nitrogen, wherein the 5-6 membered heterocyclic group is fully saturated or partially unsaturated and is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; amino; N—(C₁-C₇-alkyl)amino; or N,N-di(C₁-C₇-alkyl)amino group;
m denotes an integer from 0 to 4;
R¹ denotes hydrogen, halo, C₁-C₇ alkyl which is unsubstituted or substituted by 1 to 3 groups selected from halo, hydroxy, C₁-C₄ alkoxy, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; C₂-C₇ alkenyl which is unsubstituted or substituted by 1 to 3 halo atoms; C₂-C₇ alkinyl; C₁-C₄ alkyloxycarbonyl, monocyclic or bicyclic aryl group having from 6 to 10 carbon atoms and alternating double bonds between carbon atoms, wherein the monocyclic or bicyclic aryl group is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; C₄-C₁₂ mocyclic or bicyclic heteroaryl ring system which is aromatic or partially aromatic containing at least one heteroatom selected from oxygen, sulphur and nitrogen, wherein C₄-C₁₂ monocyclic or bicyclic heteroaryl ring system is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; 5-6 membered heterocyclic group containing at least 1 heteroatom selected from oxygen, sulphur and nitrogen, wherein the 5-6 membered heterocyclic group is fully saturated or partially unsaturated and is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; hydroxy; hydroxy-C₂-C₇ alkenyl; hydroxy-C₂-C₇ alkinyl; C₁-C₇ alkoxy; thiol; thio-C₂C₇ alkenyl; thio-C₂C₇ alkinyl; C₁-C₇ alkylthiol; amino; N—(C₁-C₇-alkyl)amino; N,N-di(C₁-C₇-alkyl)amino; C₁-C₇ alkylamino; amino-C₂-C₇ alkenyl; amino-C₂-C₇ alkinyl; amino-C₁-C₇ alkoxy; C₁-C₇ alkanoyl; aroyl, oxo-C₁-C₇ alkyl; C₁-C₇ alkanoyloxy; carboxy; C₁-C₇ alkyloxycarbonyl wherein alkyl has the meaning as defined above, aryloxycarbonyl, wherein aryl has the meaning as defined above; carbamoyl; N—(C₁-C₇-alkyl)carbamoyl; N,N-di(C₁-C₇-alkyl)carbamoyl; cyano; cyano-C₁-C₇ alkyl; sulfonyl; C₁-C₇ alkylsulfonyl; sulfinyl; C₁-C₇ alkylsulfinyl; nitro ;or a substituent of the formula II, wherein the symbols A and m have the meaning as defined above;
and pharmacologically acceptable esters, salts and solvates thereof.

2. Compound according to claim 1, wherein X is —O—.

3. Compound according to claim 2, wherein the symbol m is 1, 2 or 3.

4. Compound according to claim 2, wherein A is —N(CH₃)₂, pyrrolidin-1-yl or phenyl.

5. The compound of formula I according to claim 1 selected from the group consisting of:
10-benzyloxy-8-oxa-1-thia-dibenzo[e,h]azulene-2-carboxylic acid ethyl ester;
10-(3-dimethylamino-propoxy)-8-oxa-1-thia-dibenzo[e,h]azulene-2-carboxylic acid ethyl ester;
10-(2-dimethylamino-ethoxy)-8-oxa-1-thia-dibenzo[e,h]azulene-2-carboxylic acid ethyl ester;
10-(2-pyrrolidin-1-yl-ethoxy)-8-oxa-1-thia-dibenzo[e,h]azulene-2-carboxylic acid ethyl ester;
11-benzyloxy-8-oxa-1-thia-dibenzo[e,h]azulene-2-carboxylic acid ethyl ester;
11-(3-dimethylamino-propoxy)-8-oxa-1-thia-dibenzo[e,h]azulene-2-carboxylic acid ethyl ester;
10-(3-dimethylamino-propoxy)-8-oxa-1-thia-dibenzo[e,h]azulene-2-carboxylic acid;
(10-benzyloxy-8-oxa-1-thia-dibenzo[e,h]azulene-2-yl)-methanol;
(11-benzyloxy-8-oxa-1-thia-dibenzo[e,h]azulene-2-yl)-methanol;
[10-(3-dimethylamino-propoxy)-8-oxa-1-thia-dibenzo[e,h]azulene-2-yl]-methanol;
[11-(3-dimethylamino-propoxy)-8-oxa-1-thia-dibenzo[e,h]azulene-2-yl]-methanol;
[3-(10-benzyloxy-8-oxa-1-thia-dibenzo[e,h]azulene-2-ylmethoxy)-propyl]-dimethyl-amine;
[3-(11-benzyloxy-8-oxa-1-thia-dibenzo[e,h]azulene-2-ylmethoxy)-propyl]-dimethyl-amine;
{3-[2-(3-dimethylamino-propoxymethyl)-8-oxa-1-thia-dibenzo[e,h]azulene-10-yloxy]-propyl}-dimethyl-amine;
{3-[2-(3-dimethylamino-propoxymethyl)-8-oxa-1-thia-dibenzo[e,h]azulene-11-yloxy]-propyl}-dimethyl-amine;
10-(3-dimethylamino-propoxy)-8-oxa-1-thia-dibenzo[e,h]azulene-2-carboxylic acid 3-dimethylamino-propyl ester; and
dimethyl-[3-(8-oxa-1-thia-dibenzo[e,h]azulene-10-yloxy)-propyl]-amine.

6. A process for the preparation of a compound of the formula I 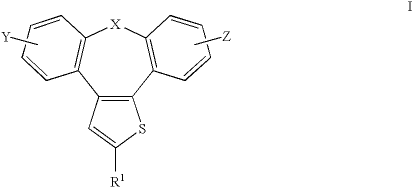
wherein
X denotes a hetero atom —O— or —S—;
Y is H;
Z is a fragment of the formula II
—O—(CH₂)ₘ-A II
wherein
A denotes hydrogen, an C₁-C₇ alkyl which is unsubstituted or substituted by 1 to 3 groups selected from halo, hydroxy, C₁-C₄ alkoxy, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; C₂-C₄ alkenyl which is unsubstituted or substituted by 1 to 3 halo atoms; C₂-C₇ alkinyl; monocyclic or bicyclic aryl group having from 6 to 10 carbon atoms and alternating double bonds between carbon atoms, wherein the monocyclic or bicyclic aryl group is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; C₄-C₁₂ monocyclic or bicyclic heteroaryl ring system which is aromatic or partially aromatic containing at least one heteroatom selected from oxygen, sulphur and nitrogen, wherein C₄-C₁₂ monocyclic or bicyclic heteroaryl ring system is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; 5-6 membered heterocyclic group containing at least 1 heteroatom selected from oxygen, sulphur and nitrogen, wherein the 5-6 membered heterocyclic group is fully saturated or partially unsaturated and is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; amino; N—(C₁-C₇-alkyl)amino; or N,N-di(C₁-C₇-alkyl)amino group;
m denotes an integer from 1 to 4;
R¹ denotes hydrogen, halo, C₁-C₇ alkyl which is unsubstituted or substituted by 1 to 3 groups selected from halo, hydroxy, C₁-C₄ alkoxy, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; C₂-C₇ alkenyl which is unsubstituted or substituted by 1 to 3 halo atoms; C₂-C₇ alkinyl; C₁-C₄ alkyloxycarbonyl, monocyclic or bicyclic aryl group having from 6 to 10 carbon atoms and alternating double bonds between carbon atoms, wherein the monocyclic or bicyclic aryl group is unsubstituted or is substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; C₄-C₁₂ monocyclic or bicyclic heteroaryl ring system which is aromatic or partially aromatic containing at least one heteroatom selected from oxygen, sulphur and nitrogen, wherein C₄-C₁₂ monocyclic or bicyclic heteroaryl ring system is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; 5-6 membered heterocyclic group containing at least 1 heteroatom selected from oxygen, sulphur and nitrogen, wherein the 5-6 membered heterocyclic group is fully saturated or partially unsaturated and is unsubstituted or substituted by 1 or 2 substituents selected from halo, C₁-C₄ alkyl, trifluoromethyl, cyano, nitro, hydroxy, C₁-C₄ alkoxy, C₁-C₄ alkyloxycarbonyl, thiol, C₁-C₄ alkylthio, amino, N—(C₁-C₄) alkylamino, N,N-di(C₁-C₄-alkyl)-amino, sulfonyl, C₁-C₄ alkylsulfonyl, sulfinyl and C₁-C₄ alkylsulfinyl; hydroxy; hydroxy-C₂-C₇ alkenyl; hydroxy-C₂-C₇ alkinyl; C₁-C₇ alkoxy; thiol; thio-C₂C₇ alkenyl; thio-C₂C₇ alkinyl; C₁-C₇ alkylthiol; amino; N—(C₁-C₇-alkyl)amino; N,N-di(C₁-C₇-alkyl)amino; C₁-C₇ alkylamino; amino-C₂-C₇ alkenyl; amino-C₂-C₇ alkinyl; amino-C₁-C₇ alkoxy; C₁-C₇ alkanoyl; aroyl; oxo-C₁-C₇ alkyl; C₁-C₇ alkanoyloxy; carboxy; C₁-C₇ alkyloxycarbonyl, wherein alkyl has the meaning as defined above; aryloxycarbonyl, wherein aryl has the meaning as defined above; carbamoyl; N—(C₁-C₇-alkyl)carbamoyl; N,N-di(C₁-C₇-alkyl)carbamoyl; cyano; cyano-C₁-C₇ alkyl; sulfonyl; C₁-C₇ alkylsulfonyl; sulfinyl; C₁-C₇ alkylsulfinyl; nitro ; or a substituent of the formula II, wherein the symbols A and m have the meaning as defined above;
and pharmacologically acceptable esters, salts and solvates thereof,
characterized in that the preparation processes comprises:
a) a condensation reaction of an alcohol of the formula Illa 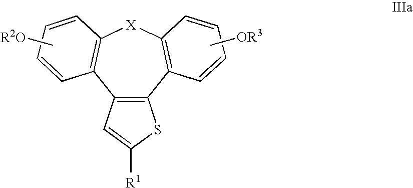
wherein at least one of the radicals R² and R³ individually denotes hydrogen and the other one a protecting group, whereas R¹ has the meaning as defined above;
or a condensation of a compound of the formula IIIb 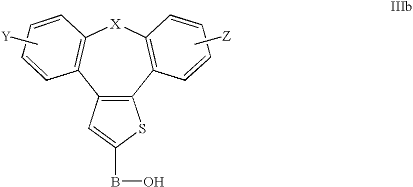
wherein B denotes a —C(O)— or —CH₂— group and Y and Z have the meaning as defined above,
with a compound of the formula IVa
L¹-(CH₂)ₘ-A IVa
wherein L¹ is a leaving group and m and A have the meaning as defined above;
b) a condensation reaction of a compound of the formula IlIc 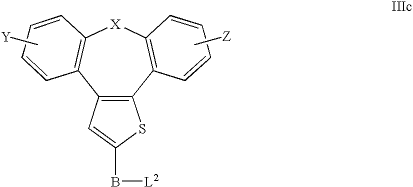
wherein B denotes a —C(O)— or —CH₂— group, L² has the individual meaning of a leaving group and Y and Z have a meaning as defined above,
with a compound of the formula IVb
HO—(CH₂)ₘ-A IVb
wherein A and m have the meaning as defined above.

7. A method of treating inflammatory diseases and conditions induced by extensive production of TNF-α and/or IL-1 comprising administering a compound as claimed in claim 1.

8. The method according to claim 7, wherein inflammatory diseases and conditions induced by extensive production of TNF-α and/or IL-1 are selected from a group consisting of rheumatoid arthritis, rheumatoid spondylitis, osteoarthritis, eczemas, psoriasis, burns caused by UV radiation, inflammatory eye diseases, Crohn's disease, ulcerative colitis and asthma.

9. The method of claim 7, wherein X is —O—.

10. The method of claim 9, wherein the symbol m is 1, 2 or 3.

11. The method of claim 9, wherein A is —N(CH₃)₂, pyrrolidin-1-yl or phenyl.

12. The method of claim 7, wherein the compound as claimed in claim 1 is selected from the group consisting of:
10-benzyloxy-8-oxa-1-thia-dibenzo[e,h]azulene-2-carboxylic acid ethyl ester;
10-(3-dimethylamino-propoxy)-8-oxa-1-thia-dibenzo[e,h]azulene-2-carboxylic acid ethyl ester;
10-(2-dimethylamino-ethoxy)-8-oxa-1-thia-dibenzo[e,h]azulene-2-carboxylic acid ethyl ester;
10-(2-pyrrolidin-1-yl-ethoxy)-8-oxa-1-thia-dibenzo[e,h]azulene-2-carboxylic acid ethyl ester;
11-benzyloxy-8-oxa-1-thia-dibenzo[e,h]azulene-2-carboxylic acid ethyl ester;
11-(3-dimethylamino-propoxy)-8-oxa-1-thia-dibenzo[e,h]azulene-2-carboxylic acid ethyl ester;
10-(3-dimethylamino-propoxy)-8-oxa-1-thia-dibenzo[e,h]azulene-2-carboxylic acid;
(10-benzyloxy-8-oxa-1-thia-dibenzo[e,h]azulene-2-yl)-methanol;
(11-benzyloxy-8-oxa-1-thia-dibenzo[e,h]azulene-2-yl)-methanol;
[10-(3-dimethylamino-propoxy)-8-oxa-1-thia-dibenzo[e,h]azulene-2-yl]-methanol;
[11-(3-dimethylamino-propoxy)-8-oxa-1-thia-dibenzo[e,h]azulene-2-yl]-methanol;
[3-(10-benzyloxy-8-oxa-1-thia-dibenzo[e,h]azulene-2-ylmethoxy)-propyl]-dimethyl-amine;
[3-(11-benzyloxy-8-oxa-1-thia-dibenzo[e,h]azulene-2-ylmethoxy)-propyl]-dimethyl-amine;
{3-[2-(3-dimethylamino-propoxymethyl)-8-oxa-1-thia-dibenzo[e,h]azulene-10-yloxy]-propyl}-dimethyl-amine;
{3-[2-(3-dimethylamino-propoxymethyl)-8-oxa-1-thia-dibenzo[e,h]azulene-11-yloxy]-propyl}-dimethyl-amine;
10-(3-dimethylamino-propoxy)-8-oxa-1-thia-dibenzo[e,h]azulene-2-carboxylic acid 3-dimethylamino-propyl ester; and
dimethyl-[3-(8-oxa-1-thia-dibenzo[e,h]azulene-10-yloxy)-propyl]-amine.

13. A pharmaceutical formulation comprising a compound as claimed in claim 1 and pharmaceutically acceptable carrier or solvent.

14. The pharmaceutical formulation of claim 13, wherein X is —O—.

15. The pharmaceutical formulation of claim 14, wherein the symbol m is 1, 2 or 3.

16. The pharmaceutical formulation of claim 14, wherein A is —N(CH₃)₂, pyrrolidin-1-yl or phenyl.

17. The pharmaceutical formulation of claim 13, wherein the compound as claimed in claim 1 is selected from the group consisting of:
10-benzyloxy-8-oxa-1-thia-dibenzo[e,h]azulene-2-carboxylic acid ethyl ester;
10-(3-dimethylamino-propoxy)-8-oxa-1-thia-dibenzo[e,h]azulene-2-carboxylic acid ethyl ester;
10-(2-dimethylamino-ethoxy)-8-oxa-1-thia-dibenzo[e,h]azulene-2-carboxylic acid ethyl ester;
10-(2-pyrrolidin-1-yl-ethoxy)-8-oxa-1-thia-dibenzo[e,h]azulene-2-carboxylic acid ethyl ester;
11-benzyloxy-8-oxa-1-thia-dibenzo[e,h]azulene-2-carboxylic acid ethyl ester;
11-(3-dimethylamino-propoxy)-8-oxa-1-thia-dibenzo[e,h]azulene-2-carboxylic acid ethyl ester;
10-(3-dimethylamino-propoxy)-8-oxa-1-thia-dibenzo[e,h]azulene-2-carboxylic acid;
(10-benzyloxy-8-oxa-1-thia-dibenzo[e,h]azulene-2-yl)-methanol;
(11-benzyloxy-8-oxa-1-thia-dibenzo[e,h]azulene-2-yl)-methanol;
[10-(3-dimethylamino-propoxy)-8-oxa-1-thia-dibenzo[e,h]azulene-2-yl]-methanol,
[11-(3-dimethylamino-propoxy)-8-oxa-1-thia-dibenzo[e,h]azulene-2-yl]-methanol;
[3-(10-benzyloxy-8-oxa-1-thia-dibenzo[e,h]azulene-2-ylmethoxy)-propyl]-dimethyl-amine;
[3-(11-benzyloxy-8-oxa-1-thia-dibenzo[e,h]azulene-2-ylmethoxy)-propyl]-dimethyl-amine;
{3-[2-(3-dimethylamino-propoxymethyl)-8-oxa-1-thia-dibenzo[e,h]azulene-10-yloxy]-propyl}-dimethyl-amine;
{3-[2-(3-dimethylamino-propoxymethyl)-8-oxa-1-thia-dibenzo[e,h]azulene-11-yloxy]-propyl}-dimethyl-amine;
10-(3-dimethylamino-propoxy)-8-oxa-1-thia-dibenzo[e,h]azulene-2-carboxylic acid 3-dimethylamino-propyl ester; and
dimethyl-[3-(8-oxa-1-thia-dibenzo[e,h]azulene-10-yloxy)-propyl]-amine.
